(19) United States
(12) Patent Application Publication
(10) Pub. No.: US 2007/0092056 A1
(43) Pub. Date: Apr. 26, 2007

(54) Method for generating CT displays in x ray computed tomography

(76) Inventors: Thomas Flohr, Uehlfeld (DE); Rainer Raupach, Adelsdorf (DE)

Correspondence Address: HARNESS, DICKEY & PIERCE, P.L.C., P.O.BOX 8910, RESTON, VA, 20195, US

(21) Appl. No.: 11/581,148

(22) Filed: Oct. 16, 2006

(30) Foreign Application Priority Data
Oct. 17, 2005 (DE) 10 2005 049 586.9

(51) Int. Cl. H05G 1/60; A61B 6/00; G01N 23/00; G21K 1/12

(52) U.S. Cl. 378/4

(57) ABSTRACT

A method is disclosed for generating CT displays in x-ray computed tomography with contrast medium support, the blooming effect being reduced by decomposing an object into three material components when scanning the object with two different energy spectra, and determining a first component and determining the material thickness thereof by segmentation. Subsequently, in at least one embodiment, the two other material components and their material thicknesses are determined on the basis of the measured attenuation values of the two spectra for each beam, and virtual absorption data with virtual absorption coefficients are constructed for the individual material components from the material strengths thus known for the different material components, and are used to reconstruct the CT display.

### PRIORITY STATEMENT

The present application hereby claims priority under 35 U.S.C. §119 on German patent application number DE 10 2005 049 586.9 filed Oct. 17, 2005, the entire contents of which is hereby incorporated herein by reference.

1. Field

The invention generally relates to a method for CT display in x-ray computed tomography. For example, it relates to one in which an object that is composed of N+1 materials or material compositions with different absorption coefficients is scanned by revolving ray fans that generate a multiplicity of scanning beams in space, with N≧2 different energy spectra, and CT displays of absorption coefficients are reconstructed from measured absorption data as a tomogram or as volume data.

2. Background

The presence of a number of materials in an object being scanned using an x ray CT method means that artifacts which can lead to misinterpretations occur in the reconstruction and the image resulting therefrom, particularly in the case of subsequent quantitative evaluations. On the one hand, there is the problem of beam hardening that is corrected during the conditioning of raw data only in an overall fashion for one material, usually water. However, since the beam hardening characteristics basically differ for different material compositions and arrangements in a scanned object, in particular a patient, such as water, bone or iodine, for example, in the case of pictures with the aid of contrast media, artifacts result in the reconstruction.

On the other hand, there is the problem of so-called blooming. When conducting CT angiographies, it is necessary to make a quantitative measurement of vessel diameters in the region of stenoses. As a rule, such stenoses, which are caused by calcified plaques, appear larger than their actual extent because of their significantly higher absorption coefficient by comparison with their surroundings, and of the filters used in the reconstruction. This complicates the correct determination of the residual volume of the vessels being considered and leads to misinterpretations.

### SUMMARY

In at least one embodiment of the invention, a method is for generating CT displays in x ray computed tomography which, especially, leads to a reduction in the blooming effect. In addition, one aim of at least one embodiment is also to take account of the beam hardening more effectively in the reconstruction on the basis of the actual facts relating to the scanned object.

The inventors have realized that it is possible, in at least one embodiment, to reduce the so-called blooming effect and at the same time to carry out an improved beam hardening correction by decomposing an object into three material components when scanning the object with two different energy spectra, a first component being determined by segmentation, and the two other material components being determined on the basis of the measured attenuation values of both spectra for each beam, and virtual absorption data with virtual absorption coefficients subsequently being constructed for the individual material components from the material thicknesses, thus known, of the different material components, and being used for a reconstruction.

Consequently, in a first step the spatial distribution of the local density of a material component is determined from a reconstructed image by using a single spectrum or a combination of the data of two spectra by segmentation. In this process, a lower or upper threshold value, or else a windowing can be used for the CT values. Alternatively, it is also possible to use a ρ/z decomposition such as described, for example, in patent application DE 101 43 131 A1 (the entire contents of which are hereby incorporated herein by reference) for such a segmentation, or the segmentation can be done by considering CT value relationships of two reconstructed CT images that have been recorded with different energy spectra. The available projection data relating to the two energy spectra, and the predetermined material thickness of the first material now permit the transirradiated material thicknesses of the two other materials to be determined. This can be implemented, for example, by a lookup procedure or by appropriately adapted functions.

Subsequently, all the material thicknesses are back calculated to form virtual pseudo-monochromatic attenuation data by using attenuation coefficients that are arbitrary in principle. The blooming effect can now be significantly reduced by selection of a fictional attenuation coefficient. This corresponds fundamentally to a type of nonlinear contrast reduction that is, however, applied not to the finished image data but to the attenuation data of the CT measurement originally present.

If, for example, the originally segmented material is to be emphasized with high contrast in the finished image, the image from the virtual attenuation data can be superposed on the originally segmented image such that either the segmented material is emphasized with high contrast, or this material can be characterized by a particular coloring. Fundamentally, if the considered materials or material components exhibit sufficiently significant differences in their attenuation coefficients, and also each individual material is reconstructed and segmented with aid of the attenuation values actually recorded, it is possible that each individual material can additionally be emphasized in a polychromatic display in a particularly striking fashion by superposing the image reconstructed from the virtual attenuation data.

In accordance with this basic idea, in at least one embodiment the inventors propose the method, known per se, for generating CT displays in x ray computed tomography, in the case of which an object, preferably a patient, that/who is composed of N+1 materials or material compositions with significantly different absorption coefficients is scanned by revolving ray fans that generate a multiplicity of scanning beams in space, with N≧2 different energy spectra, and CT displays of absorption coefficients are reconstructed from measured absorption data as a tomogram or as volume data, at least the following method steps being carried out in accordance with at least one embodiment of the invention:
a first CT display is reconstructed from the absorption data of at least one energy spectrum, and a first material or a first material composition is segmented from knowledge of its absorption coefficient,
the material thickness of the first material or of the first material composition is determined for each scanning beam in space on the basis of the first CT display,
the material thicknesses of the N other materials or material compositions are determined for each scanning beam in space by taking account of the known absorption of the first material from the N spatially identical scanning beams of different energy spectra,
a virtual attenuation value is calculated for each scanning beam in space from the N+1 known material thicknesses with the aid of newly defined absorption coefficients, and
a second CT display is reconstructed with the aid of the virtual attenuation values.

In order to reduce the blooming effect, it is advantageous when span of values of the newly defined absorption coefficients is smaller than the span of values of the absorption coefficients of the N+1 materials or material compositions. However, selecting the newly defined absorption coefficients such that the mutual spacing of values is identical or fits as far as possible is also sufficient just to reduce the blooming effect. This likewise reduces the contrast at material transitions such that the blooming effect is further reduced.

A CT display that is formed by absorption coefficients with a relatively small range of values fundamentally appears to have a lower contrast, and so individual material components also appear to be emphasized optically to lesser effect. This disadvantage can be eliminated, for example, by generating a third CT display by superposing the segmented first CT display on the second CT display.

In a particular design of the method according to at least one embodiment of the invention, the inventors further propose that at least one lookup table is made available for determining the material thicknesses of N different materials or material compositions on the basis of a known material thickness of the first material as a function of the absorption values of N energy spectra. It is possible thereby for intermediate values lacking in the lookup table to be determined by interpolation.

Such a lookup table can be determined, for example, in the following way. The absorptions A1, . . . , A_{N} in the case of the spectra S₁, . . . , S_{N} are measured for all the combinations of the material thicknesses dM₁, . . . , dM_{N+1}. The mappings F_dM₁:(dM₂, . . . , DM_{N+1})→(A₁, . . . , A_{N}) are then inverted for fixed values dM₁, this being possible on the basis of the strictly monotonic behavior of all the variables. The result is mappings G_dM₁:(A₁, . . . , A_{N})→:dM₂, . . . , dM_{N+1}) with the aid of which the remaining material thicknesses dM₂, . . . , dM_{N+1} can be calculated for material thicknesses dM₁ and N spectral measured values, and can be tabulated for fixed values of dM₁ in N N-dimensional data fields in each case. In addition to the measurement of the absorptions, the mappings F_dM₁ can also be determined by a calculation using a computer simulation.

It is to be pointed out here that the effect of a beam hardening correction is also simultaneously achieved by this pseudo-monochromatic synthesization described above.

In a further variant of the method according to at least one embodiment of the invention, the determination of the material thicknesses of N different materials or material compositions can be performed by solving a system of N nonlinear equations, preferably absorption equations, and N unknown material thicknesses by taking account of known absorption coefficients of the materials or material compositions as a function of the energy spectra.

The inventors further propose, in at least one embodiment, that the segmentation of the first material or of the first material composition is performed by setting at least one limiting value for the absorption coefficient. This can either involve a threshold value such that all the image values with an absorption coefficient above this limiting value are regarded as material-related, or it is possible to form an upper and a lower limiting value for setting as a window, or to define an upper limiting value such that all the image values below this limiting value are regarded as related to the material for the purpose of the segmentation.

Considering the important field of application of the method according to at least one embodiment of the invention, specifically CT angiography, in which a patient is scanned, the patient substantially comprising with reference to the absorption coefficients tissues that resemble water, calcium-containing bones and/or plaques and contrast media that preferably contain iodine, it is substantially calcium that can be considered as first material composition, substantially iodine that can be considered as second material component, and substantially water that can be considered as third material composition.

Moreover, it is proposed in the case of the CT display that at least one material or one material composition is assigned a specific color, it being possible here, for the purpose of improving the contrast, to carry out a segmentation for each individual material composition from the original CT data such that a corresponding image superposition can subsequently be undertaken with the segmented CT data.

The proposed method is fundamentally suitable for any type of CT units, the different energy spectra used being achieved, for example, by varying the accelerating voltage in the tube generating x radiation. It is also possible to use appropriate intermediate filters to harden the x radiation differently such that different x ray spectra are available for the scan. For these variants there is a possibility of using a CT unit with a single focus/detector system. Alternatively, it is also possible to use a CT unit with a number of focus/detector systems, each focus/detector system preferably being used to scan another energy spectrum. If, for example, a double focus/detector system with different accelerating voltages is used, it is possible by means of different filtering to use a total of at least four different spectra for the scan, the scanned object as a whole being capable of decomposition into five material compositions.

It may be pointed out that, given sufficiently different absorption coefficients of the materials, it is also within the scope of the invention to use the primary segmentation to segment these different materials per se and also to determine N+J materials on the basis of the knowledge of the position and mixture of these materials in the scanned object with one scan having N different energy spectra, J corresponding to the number of the materials that can be segmented in the primary decomposition.

### BRIEF DESCRIPTION OF THE DRAWINGS

The invention is described in more detail below using an example embodiment with the aid of the figures, only the features required to understand the invention being illustrated. Identical elements are provided in the various figures with the same reference symbols, these having the following meaning: 1: CT system; 2: first x ray tube; 3: first detector; 4: second x ray tube; 5: second detector; 6: gantry housing; 7: patient; 8: patient couch; 9: system axis; 10: control and arithmetic logic unit; 11: absorption data of the first spectrum S1; 12: absorption data of the second spectrum S2; 13: reconstruction for segmentation; 14: segmentation; 15: inverse reconstruction (=forward projection); 16: material decomposition; 17: data synthesization; 18: image reconstruction; 19: CT image I; 20: superposition; 21: superposed CT image I′; 22: vessel with plaques and contrast medium; 23: vessel with contrast medium; A′: virtual absorption; A_{y}: absorption data of the spectrum S_{y}; dM₁: material thickness of the material Mₓ; Mₓ: material component ₓ; Prg₁: computer programs; S_{y}: energy spectrum.

In detail:

FIG. 1 shows a 3D illustration of a computed tomography system having two focus/detector systems for carrying out the method according to an embodiment of the invention,

FIG. 2 shows a schematic flowchart of the method according to an embodiment of the invention,

FIG. 3 shows a schematic flowchart of the method according to an embodiment of the invention with additional superposition of a segmented image, and

FIGS. 4-8 show simulation of imaging with the method according to an embodiment of the invention.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

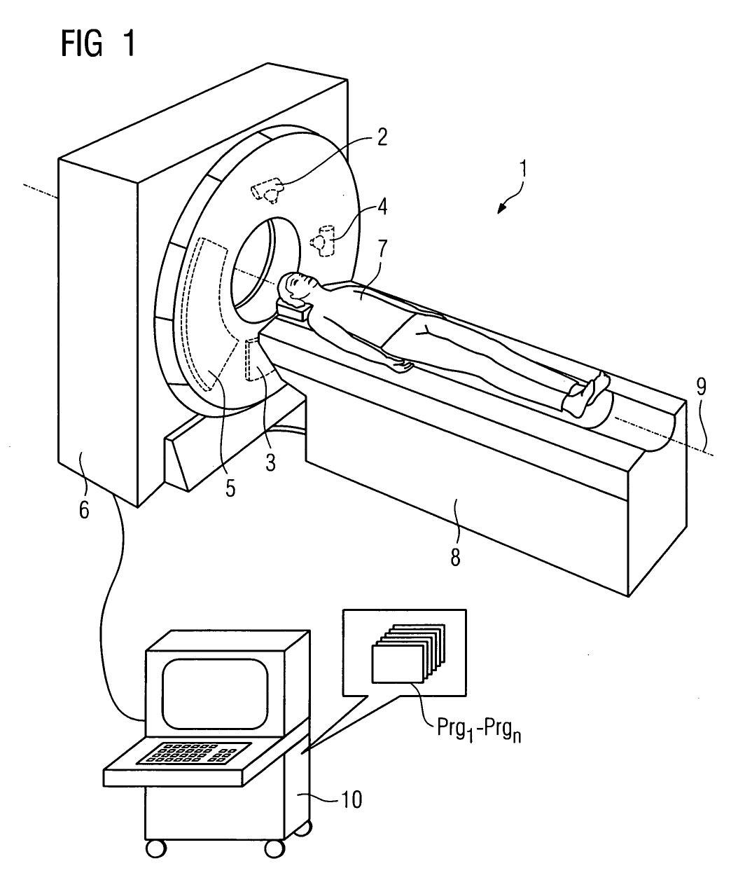

FIG. 1 shows an exemplary computed tomography system 1 having two focus/detector systems including a first x ray tube 2 with an opposite detector 3, and a second x ray tube 4 with the opposite detector 5. Both focus/detector systems can be operated with different operating voltages in order to carry out the method according to the invention, or can have different filter attachments.

For the investigation, the patient 7 is pushed with the aid of the displaceable patient couch 8 along the system axis 9 in a sequential or continuous fashion through the beam path of the two focus/detector systems that are arranged on a gantry (not visible here) in the gantry housing 6, and is scanned in the process by two radiation fans with a different energy spectrum. In the process, the detectors 3, 5 opposite the x ray tubes 2, 4 acquire the attenuation of the x radiation over the entire energy range, that is to say not for specific energies. The CT system 1 is controlled by the control and arithmetic logic unit 10 in which the data collection and reconstruction including the method according to an embodiment of the invention are undertaken. This purpose is served by programs Prg₁-Prgₙ that map the method steps according to an embodiment of the invention.

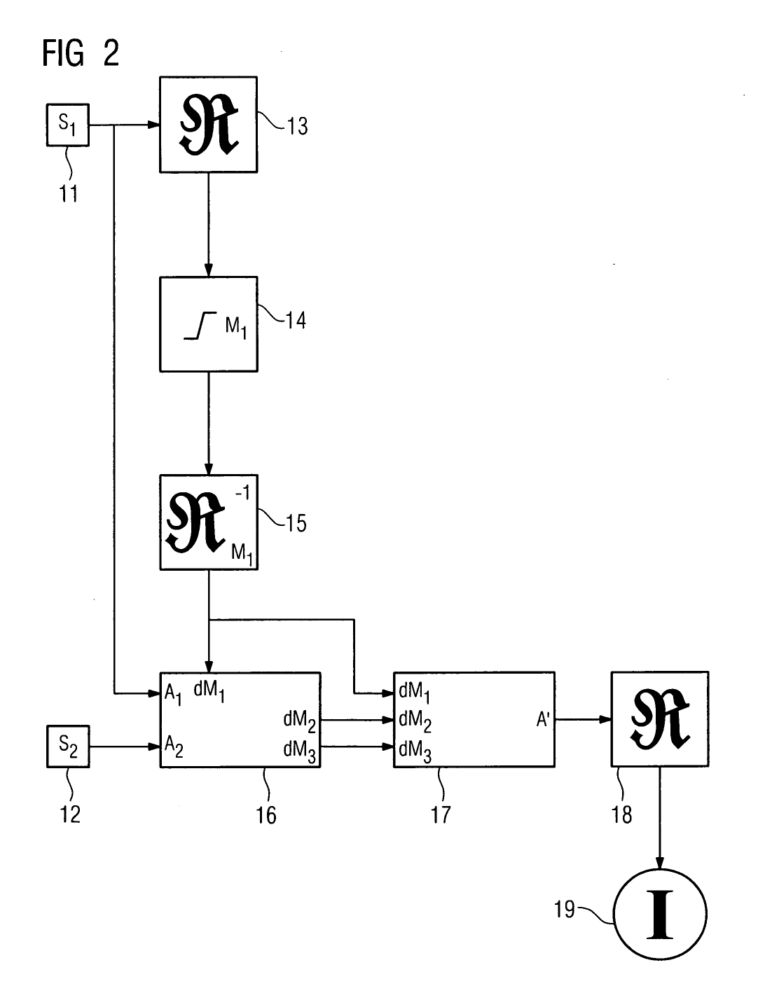

FIG. 2 shows a simple example of the method according to an embodiment of the invention in the case of which two spectra S₁ and S₂, illustrated by the boxes 11 and 12, are used in order to scan an object, in particular a patient, preferably for a CT angiography. The absorption data A₁ recorded by the spectrum S₁ are fed to a reconstruction 13. This reconstruction is carried out with relatively steep filters without particular regard to the existing image noise such that the image data obtained can be fed to a subsequent segmentation 14 in which the CT image values, which correspond to a first material or a first material composition M₁, are segmented.

The penetrated material thickness dM₁ is now determined in step 15 for each beam in space through the object, on the basis of the segmented data. On the basis of the knowledge of this material thickness dM₁ and of the absorptions A₁ and A₂, it can now be determined from the x ray spectra S₁ and S₂, respectively, how high the material thicknesses dM₂ and dM₃ turn out to be for the respectively considered x ray beam in space. It is possible to this end to make use, for example, of a lookup table that has been recorded by measuring the absorption of different material thicknesses for the three materials and been calculated by inverting the mapping (dM₂, dM₃)→(A₁, A₂) for fixed dM₁.

Thus, the material thicknesses dM₂ and dM₃ are known in method step 16 as a result, the material thickness dM₁ already being present from the inverted reconstruction 15. Starting from these material thicknesses dM₁ to dM₃ that are now known, each individual material M₁ to M₃ is accorded a virtual absorption coefficient μ₁′ to μ₃′ in method step 17 such that a virtual absorption A′ can be calculated for each individual scanning beam on the basis of the known material thicknesses, where A′=μ₁′*dM₁+μ₂′*dM₂+μ₃′*dM₃. Thus, this method can be used to calculate virtual projections that can subsequently be reconstructed in method step 18 to form CT image data or CT volume data. Since the selection of the virtual absorption coefficients is free, these can be selected such that the jumps in contrast that appear in the CT image are moderated, in particular those between iodine and bone, and thereby the blooming effect that is produced by very strong jumps in contrast and simultaneously relatively weak filtering during the reconstruction is sharply diminished.

The result is that a CT display 19 that corresponds with reference to its gray scale to the selected, virtual absorption coefficients.

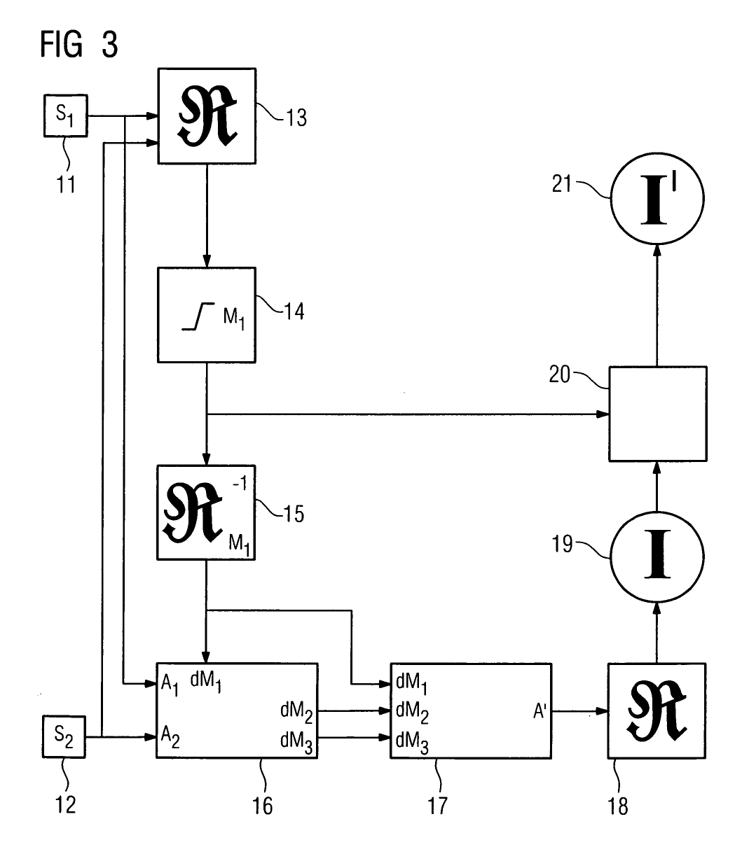

Since such a display with diminished spreading of the absorption coefficients has a diminished contrast, the inventors further propose that in addition the reconstructed image is superposed on the data obtained in the segmentation such that a display that can be interpreted more easily is produced with reference to the selected material or the material component M₁. Such a method is illustrated by way of example in FIG. 3.

The method fundamentally corresponds to the method in FIG. 2, but in this case the two spectra S₁ and S₂ have been weighted in accordance with their used dose weights, combined and used for the reconstruction in method step 13. A noise-optimized weighting in the case of which the weights of the data S₁ and S₂ are selected in accordance with their dose proves to be advantageous. The segmentation in step 14, and the subsequent inverted reconstruction in step 15 remain the same, likewise the downstream method steps 16, 17, 18 and 19. What is new in this method is that the reconstructed image 19 is superposed on the segmented image after the method step 14 in a new method step 20, and a new CT image 21 is produced that is more strongly designed with reference to the contrast. Since the filtering in method step 14 turns out to be substantially steeper than in method step 18, the blooming effect is greatly reduced here such that the actual thickness of the material M₁ is represented by the segmented image with great accuracy.

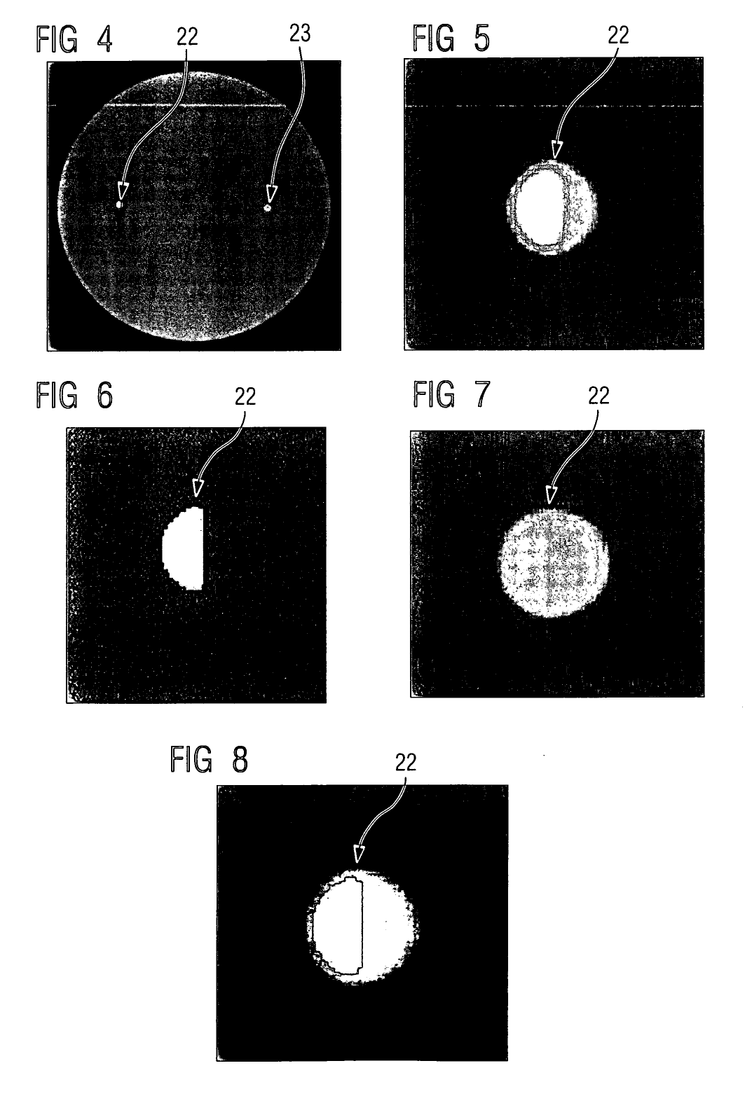

FIGS. 4 to 8 show by way of example the effect of the method according to the invention on a virtual phantom. This virtual phantom is illustrated in FIG. 4 and includes a cylindrical water phantom in which there are arranged two vessels 22 and 23 through which a contrast medium flows. The left-hand vessel 22 additionally has a calcification over half the volume. The CT display of FIG. 4 was calculated with the aid of a normal reconstruction method.

FIG. 5 illustrates an enlargement of this left-hand vessel 22, half filled with plaque, for a normal reconstruction. It is to be seen that an enlargement of the volume of the calcification is clearly discernible owing to the blooming effect.

FIG. 6 shows the result of the segmentation in accordance with the method step 14 in FIGS. 2 and 3, in which a clearly delimited, semicircular calcium segment is to be discerned. The result of the reconstruction from method step 18 of FIGS. 2 and 3 is illustrated in FIG. 7, a substantially lesser bandwidth having been selected here for the virtual absorption coefficients than is actually present. The vessel is also correspondingly not falsely enlarged by the blooming effect. However, it is to be seen that the contrast is insufficient—at least in the way in which the virtual absorption coefficients have been selected here—for diagnosing the calcification. Consequently, a superposition of FIGS. 6 and 7 is undertaken in the method according to the invention. This superposition is illustrated in FIG. 8. A clear delimitation of the plaque present in the vessel is now to be seen here, this volume not being enlarged by blooming effects, and thus permitting a substantially improved diagnosis.

Thus, overall an embodiment of this invention results in a method for generating CT displays in x ray computed tomography in a fashion supported by contrast media, the blooming effect being reduced by virtue of the fact that during scanning of an object with two different energy spectra S₁ and S₂ the object is decomposed into three material components M₁, M₂ and M₃, and a first component M₁ and the material thickness dM₁ thereof are determined by segmentation. Subsequently, the two other material components M₂ and M₃ and their material thicknesses dM₂ and dM₃ are determined for each beam on the basis of the measured attenuation values A₁ and A₂ of the two spectra S₁ and S₂, and virtual absorption data A′ with virtual absorption coefficients are constructed for the individual material components M₁, M₂ and M₃ from the material thicknesses dM₁, dM₂ and dM₃, thus known, of the different material components M₁, M₂ and M₃, and are used for the reconstruction of the CT display to be prepared.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method-of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for generating CT displays in x-ray computed tomography, comprising:
scanning an object, composed of N+1 materials or material compositions with different absorption coefficients, by revolving ray fans that generate a multiplicity of scanning beams in space, with N≧2 different energy spectra;
reconstructing a first CT display from the absorption data of at least one energy spectrum, a first material or a first material composition being segmented from knowledge of its absorption coefficient;
determining a material thickness of the first material or of the first material composition, for each scanning beam in space on the basis of the first CT display;
determining the material thicknesses, of the N other materials or material compositions for each scanning beam in space, by taking account of the known absorption of the first material from the N spatially identical scanning beams of different energy spectra;
calculating a virtual attenuation value for each scanning beam in space from the N+1 known material thicknesses with the aid of newly defined absorption coefficients; and
reconstructing a second CT display with the aid of the virtual attenuation values.

2. The method as claimed in claim 1, wherein the span of values of the newly defined absorption coefficients is smaller than the span of values of the absorption coefficients of the N+l materials or material compositions.

3. The method as claimed in claim 1, wherein a third CT display is generated by superposing the segmented first CT display on the second CT display.

4. The method as claimed in claim 1, wherein at least one lookup table is made available for determining the material thicknesses of N other different materials or material compositions on the basis of a known material thickness of the first material as a function of the absorption values of N energy spectra.

5. The method as claimed in claim 4, wherein missing intermediate values in the lookup table are determined by interpolation.

6. The method as claimed in claim 4, wherein the lookup table is determined by absorption measurements with the aid of the energy spectra used at different material thicknesses of the considered materials or material compositions.

7. The method as claimed in claim 4, wherein the lookup table is determined by calculating the absorption of the energy spectra used at different material thicknesses of the considered materials or material compositions.

8. The method as claimed in claim 1, wherein the determination of the material thicknesses of N other different materials or material compositions is performed by solving a system of equations with N absorption equations and N unknown material thicknesses by taking account of known absorption coefficients of the materials or material compositions as a function of the energy spectra.

9. The method as claimed in claim 1, wherein the segmentation of the first material or of the first material composition is performed by setting at least one limiting value for the absorption coefficient.

10. The method as claimed in claim 9, wherein the segmentation of the first material or of the first material composition is performed by setting an upper and a lower limiting value for the absorption coefficient.

11. The method as claimed in claim 1, wherein the first material composition consists substantially of calcium.

12. The method as claimed in claim 1, wherein the second material composition consists substantially of iodine.

13. The method as claimed in claim 1, wherein the third material composition consists substantially of water.

14. The method as claimed in claim 1, wherein a color is assigned to at least one material or one material composition in the CT display.

15. The method as claimed in claim 1, wherein the different energy spectra used are generated by separate focus/detector systems.

16. The method as claimed in claim 1, wherein the different energy spectra used are generated by a sing-l-e focus/detector system.

17. The method as claimed in claim 2, wherein a third CT display is generated by superposing the segmented first CT display on the second CT display.

18. The method as claimed in claim 5, wherein the lookup table is determined by absorption measurements with the aid of the energy spectra used at different material thicknesses of the considered materials or material compositions.

19. The method as claimed in claim 5, wherein the lookup table is determined by calculating the absorption of the energy spectra used at different material thicknesses of the considered materials or material compositions.

20. A computer readable medium including program segments for, when executed on a computer device of a radiological system, causing the radiological system to implement the method of claim 1.
